(19) United States
(12) Patent Application Publication
(10) Pub. No.: US 2023/0079814 A1
(43) Pub. Date: Mar. 16, 2023

(54) IMAGING SYSTEM

(71) Applicants: Jacques VAN DAM, Los Angeles, CA (US); Daniel Joseph RACHLIN, San Jose, CA (US); Venkata Vishnu GURUKULA, Mountain House, CA (US)

(72) Inventors: Jacques VAN DAM, Los Angeles, CA (US); Daniel Joseph RACHLIN, San Jose, CA (US); Venkata Vishnu GURUKULA, Mountain House, CA (US)

(21) Appl. No.: 17/944,648

(22) Filed: Sep. 14, 2022

Related U.S. Application Data
(60) Provisional application No. 63/244,212, filed on Sep. 14, 2021.

(51) Int. Cl. A61B 5/00

(52) U.S. Cl. CPC A61B 5/6861; A61B 5/0084; A61B 5/687

(57) ABSTRACT

An imaging system includes a tether, a tapered end portion coupling the tether to an imaging capsule, the imaging capsule comprising a camera lens, and at least one fluid wicking element, positioned on the imaging system proximate to the camera lens of the imaging capsule to enable the fluid wicking element to wick liquid away from the camera lens. In some embodiments, the fluid wicking element of the imaging system further functions as an alignment element for the imaging system to enable a positioning of the imaging system in an opening.

### CROSS-REFERENCE TO RELATED APPLICATIONS

This application claims priority to and the benefit of U.S. Provisional Patent Application Ser. No. 63/244,212, filed Sep. 14, 2021, which is incorporated herein by reference in its entirety.

### BACKGROUND

### Field

Embodiments described herein generally relate to an improved imaging system and more particularly to an improved esophageal imaging system.

### Description of the Related Art

There are several major areas of concern with respect to creating an esophageal imaging device that can be used to serve a broad population during, for example, routine visits to a primary care physician's office. These factors have been underappreciated in the state of the art. Each of the following is of importance: 1) speed of exam; 2) patient comfort including the elimination of anxiety associated with passage of the device from the mouth and down the throat; 3) ability to optimize the view, especially of the lower anatomy; 4) avoidance of discomfort induced by retrieving the device; 5) compatibility with a low cost and single use (disposable) design.

Current approaches fail to meet the requirements for serving a broad population for diagnostic screening. For example, capsule endoscopy, which involves swallowing a pill-shaped device that captures and transmits images wirelessly, avoids the discomfort associated with a tether, but encounters significant challenges in assuring that the desired view of the esophagus is captured. These devices have no mechanically coupled external means of control, and they make only a single pass through the anatomy of interest—there may be no “second chance.” There are currently very limited means to adapt the device to conditions such as poor alignment or positioning and obstructed view.

### SUMMARY

Embodiments of an improved imaging device/system, such as an esophageal imaging system, are provided herein. In some embodiments, an imaging system includes a tether, a tapered end portion coupling the tether to an imaging capsule, the imaging capsule comprising a camera lens, and at least one fluid wicking element, positioned on the imaging system proximate to the camera lens of the imaging capsule to enable the fluid wicking element to wick liquid away from the camera lens.

In some embodiments, the fluid wicking element uses at least one of a wicking property of a material of the fluid wicking element or gravity to wick liquid away from the camera lens. In some embodiments, wicked fluid can accumulate on the fluid wicking element and be drawn off of the fluid wicking element by gravity. In some embodiments, fluid accumulated on the fluid wicking element is drawn off by contact with a surrounding absorbent material. In some embodiments, the fluid wicking element of the imaging system further functions as an alignment element for the imaging system.

In some embodiments, the imaging system further includes at least one alignment element for aligning the imaging system when inserted in an opening. In some embodiments, the opening comprises a human esophagus. In some embodiments, the at least one alignment element comprises three alignment elements to center the imaging system in the opening. In some embodiments, the at least one alignment element is configured as a loop having at least two attachments to components of the imaging system. In some embodiments, the at least one alignment element comprises fluid wicking properties. In some embodiments, the fluid wicking element comprises at least one of a hydrophilic or lubricious coating.

In some embodiments, an imaging system includes a tether, a tapered end portion coupling the tether to an imaging capsule, the imaging capsule comprising a camera lens, and at least one alignment element attached on the imaging system to enable a positioning of the imaging system in an opening. In some embodiments, the imaging system further includes a palatant, wherein the at least one alignment element is attached to the palatant. In some embodiments, the at least one alignment element comprises fluid wicking properties. In some embodiments, the at least one alignment element is positioned on the imaging system proximate to the camera lens of the imaging capsule to enable the at least one alignment element comprising the fluid wicking properties to wick liquid away from the camera lens. In some embodiments, the imaging system further includes a restraining element to collapse the at least one alignment element. In some embodiments, the at least one alignment element is collapsed by the restraining element to enable placing the imaging system into an opening. In some embodiments, the restraining element is removed and the at least one alignment element expands after entering the opening. In some embodiments, the at least one alignment element is constructed of materials having such fine proportions that forces exerted on surfaces of the opening by the at least one alignment element are insufficient to impede the passage of the imaging system through the opening when the imaging system is pulled by gravity.

Other and further embodiments in accordance with the present principles are described below.

### BRIEF DESCRIPTION OF THE DRAWINGS

So that the manner in which the recited features of the present principles can be understood in detail, a more particular description of the principles, briefly summarized above, may be had by reference to embodiments, some of which are illustrated in the appended drawings. It is to be noted, however, that the appended drawings illustrate only typical embodiments in accordance with the present principles and are therefore not to be considered limiting of its scope, for the principles may admit to other equally effective embodiments.

FIG. 1A depicts a high-level block diagram of an imaging system including a drain wick in accordance with an embodiment of the present principles.

FIG. 1B depicts a high-level block diagram of an imaging system including multiple drain wicks and a connecting strut in accordance with an embodiment of the present principles

FIG. 2 depicts a high-level block diagram of an imaging system in accordance with an alternate embodiment of the present principles.

FIG. 3 depicts a high-level block diagram of an imaging system including alignment extensions in accordance with an alternate embodiment of the present principles.

FIG. 4 depicts an imaging system comprising radiating alignment extension(s) at a first stage of descension down a tube in accordance with an embodiment of the present principles.

FIG. 5 depicts an imaging system comprising radiating alignment extension(s) at a second stage of descension down a tube in accordance with an alternate embodiment of the present principles.

FIG. 6 depicts a high-level block diagram of an imaging system including alignment loops in accordance with an embodiment of the present principles.

FIG. 7 depicts a high-level block diagram of an imaging system including alignment extension which can also function as drain wicks in accordance with an embodiment of the present principles.

FIG. 8 depicts a high-level block diagram of an imaging system including drain wicks attached to looped alignment extensions in accordance with an embodiment of the present principles.

FIG. 9 depicts a high-level block diagram of an imaging system including proximally located alignment extensions in accordance with an embodiment of the present principles.

FIG. 10 depicts a high-level block diagram of an imaging system including distally located alignment extensions in accordance with an embodiment of the present principles

To facilitate understanding, identical reference numerals have been used, where possible, to designate identical elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments described herein relate to an improved imaging device, such as an esophageal imaging system. While the concepts of the present principles are susceptible to various modifications and alternative forms, specific embodiments thereof are shown by way of example in the drawings and are described in detail below. It should be understood that there is no intent to limit the concepts of the present principles to the particular forms disclosed. On the contrary, the intent is to cover all modifications, equivalents, and alternatives consistent with the present principles and the appended claims. For example, although embodiments of the present principles will be described primarily with respect to particular improvements, such teachings should not be considered limiting.

### Drain Wick

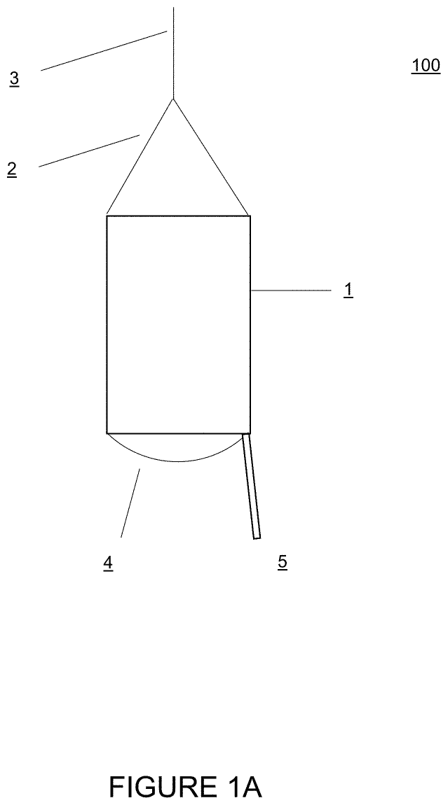

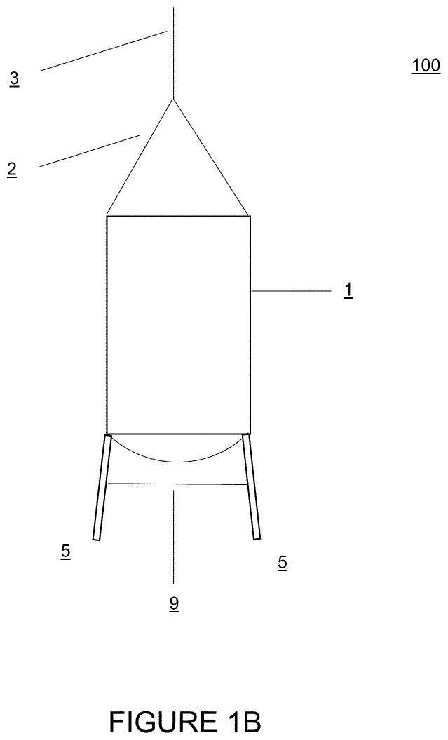

FIG. 1A depicts a high-level block diagram of an imaging system in accordance with an embodiment of the present principles. The imaging system 100 of FIG. 1A illustratively comprises an esophageal imaging capsule including an imaging capsule 1 having a tapered first end 2 ending in a tether 3, a camera lens 4 on a second end, and a drain wick attached to the imaging capsule 1 near the camera lens 4. In some embodiments, the drain wick 5 can comprise a filament with hydrophilic properties that tends to attract droplets of liquid (e.g., water) that may otherwise stay attached the lens. That is, is some embodiments, the drain wick 5 can be used to attract liquid droplets away from and off of the lens 4. Gravity and wicking can both be implemented to pull the liquid droplets along the wick where the liquid droplets can either drop off freely or be pulled away by making contact with an absorbent material, such as a tissue. In some embodiments, the drain wick 5 can be flared outward to have minimal impact on the field of view of the camera lens 4.

In some embodiments, multiple drain wicks can be attached to an imaging system of the present principles. For example, FIG. 1B depicts a high-level block diagram of an imaging system including multiple drain wicks 5 and a connecting strut 9 in accordance with an embodiment of the present principles. The multiple drain wicks 5 can be spaced at equal angles. In some embodiments, a filament design of the drain wicks can be expanded to a widened form, creating an inverted funnel or other such form that can subtend a full 360 or less degrees. In some embodiments, the multiple drain wicks can be connected with at least one strut 9, as depicted in FIG. 1B, that ensure a proper degree of flaring of portions of the drain wick away from a central axis and/or can help preserve some rigidity to the structure of the multiple drain wicks so that the multiple drain wicks do not collapse.

In some embodiments, a drain wick of the present principles can comprise a circular cross-section fiber or, alternatively a length of film with a rectangular cross-section. The attachment of such a drain wick can be to the side of an imaging system of the present principles. Alternatively, the attachment of the drain wick can be an integrated component of an imaging capsule an imaging system of the present principles.

In some embodiments, in imaging system of the present principles can include a thin metal or polymer shell that encloses at least one of electrical circuitry, a camera, lens assembly and interconnects. The space between the camera lens and the inner surface of the shell can include a location from which the drain wick can emerge and can be created as an extension of a flexible printed circuit board, typically comprising polyimide.

In some embodiments, by using a polymer such as nylon, polyester and other possibilities, an imaging system of the present principles can retain some degree of flexibility and be of minimal impact upon patient comfort when ingested. In some embodiments, the polymers or other materials used to construct the drain wick may not be hydrophilic. In such embodiments, a hydrophilic coating can be applied to at least the drain wick to obtain the desired hydrophilic properties. That is, in some embodiments the hydrophilic coating can also be applied to a substantial portion of or to the entire imaging capsule 1, the tapered end portion 2, and/or the tether 3, so that a single process can be implemented to add the hydrophilic coating to the entirety of the drain wick 5.

Hydrophilic and/or lubricious coatings applied to components of an imaging system of the present principles offer less friction and greater ease in travel of the imaging system, for example, down the esophagus, and greater ease in retrieval of the imaging system from a patient's body, significantly enhancing the ease of procedures performed with an imaging system and further improve patient comfort.

In accordance with the present principles, the drain wick 5 is located in proximity to the camera lens 4 such that droplets of liquid (e.g., water) that collect on the camera lens 4 will be absorbed by the drain wick 5, which through wicking and/or the force of gravity can pull the liquid away from the camera lens 4. That is, in some embodiments, gravity enables fluid to collect on a portion (e.g., an end) of the drain wick and reach a concentration at which the force of gravity on the weight of the fluid causes the fluid to fall away from the drain wick. By positioning a drain wick near a camera lens at the bottom of an imaging system in accordance with the present principles, multiple wicking iterations can occur without saturating the drain wick 5 because the liquid on the drain wick 5 is pulled down by gravity to at or near an end of the drain wick and drop off. Alternatively or in addition, in some embodiments a portion (e.g., an end) of the drain wick can be contacted by an absorbent material, such as a tissue, thus pulling the liquid off of the surface of the drain wick 5.

In some embodiments of the present principles, a drain wick can include an absorbent layer such as paper or methylcellulose to draw water away water from the surface of a camera lens via a combination of gravity and capillary wicking and/or to assist in keeping the drain wick dry. The methylcellulose layer provides a homogeneous material that can be formed to provide the proper wicking and mechanical properties for liquid absorption. A range of such polymers can provided by a Porex Filtration Group.

It should be noted that pore size, material density and surface energy of the wicking structure are used to affect the speed of wicking and volume of fluid held in the structure. Of great importance is the overall degree to which fluid adheres to the drain wick—especially in competition with gravity. In some embodiments of the present principles, a drain wick is designed to release accumulated droplets of water under the influence of gravity. However, if the wick is very high performance in the sense of being incorporating very hydrophilic materials and/or has a very small pore size, the fluid can be retained by the drain wick. As such, a drain wick of the present principle is designed to have sufficient hydrophilic properties to drain the fluid away from a camera lens, but not exceed the point where fluid cannot drip away from a suspended drain wick via the force of gravity. Examples of materials that exhibit a good balance in this regard can be found with common coffee filter paper and kitchen paper towels, however such materials may not have sufficient shape memory. If the drain wick does not hold its form, the wick may cling to the camera lens or to the side of the imaging capsule, negating the purpose of the drain wick.

In accordance with the present principles, the use of a composite comprising multiple materials, perhaps at least one wicking and at least one not, is an approach to tuning the multiple properties of a drain wick to obtain the desired mix that minimally comprises appropriate mechanical, geometric, and wicking properties. In some embodiments, the composite can consist of a laminate, or even a twisting or braiding of filament structures.

In accordance with the present principles, mechanical properties of a drain wick are also considered. That is, shape memory of a drain wick is very important—especially when the drain wick is wet. In some embodiments, drain wicks of the present principles comprise polymer substrates. A degree of stiffness of drain wicks of the present principles is also considered. That is—the material must be sufficiently flexible in its chosen cross-section (e.g. perhaps on the order of 0.001-3.0 square mm approximately) to be of sufficient flexibility so as not to cause discomfort when swallowing. In some embodiments of the present principles, drain wicks can comprise polyethylene, polypropylene, polytetrafluoroethylene, polyvinylidene difluoride and others.

In embodiments of the present principles, the degree of hydrophilicity of a drain wick of the present principles must be properly tuned. If a wicking property of the drain wick are not strong enough, the drain wick may not be able to overpower the surface tension of the camera lens surface. In some embodiments, a camera lens can be given a hydrophobic coating to assist the drain wick in removing liquid from the camera lens. Wicking materials can be molded, formed into sheets, die-cut etc. The wicking materials of drain wicks of the present principles can be bonded thermally and in other ways that do not interfere with the desired wicking properties.

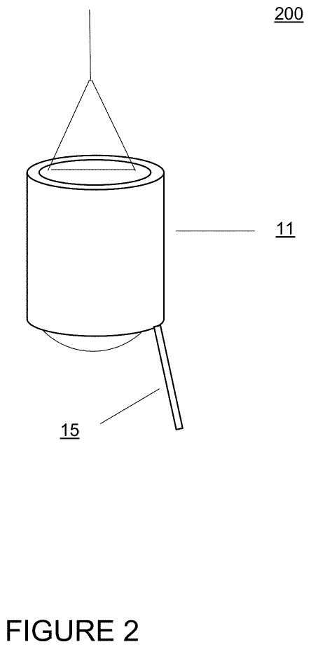

FIG. 2 depicts a high-level block diagram of an imaging system in accordance with an alternate embodiment of the present principles. The imaging system 200 of FIG. 2 illustratively comprises a cylindrical body 11 encompassing an imaging capsule (not shown). In the embodiment of FIG. 2, the cylindrical body 11 comprises a palatant. In the embodiment of FIG. 2, the drain wick 15 is attached to the palatant 11.

### Alignment

Alignment is important for an imaging system, for example, such as a tethered endoscope. Normally when free hanging, a tethered endoscope points substantially downward. However, in such an orientation, a tethered endoscope can be subjected to pendulum and spinning motions. The motion effects can cause motion blur and other artifacts in images captured by a camera (not shown) of an imaging system of the present principles. Furthermore, a tethered endoscope of the imaging system can be tilted away from a region of interest. In such systems, a question becomes how to either force the imaging system into a proper orientation, or alternatively how to maintain a consistent orientation for the imaging system.

In some embodiments, various protuberant devices/positioning mechanisms such as fins, wire loops, splaying filaments, etc. can be attached to a palatant or a tether of an imaging system of the present principles. The arms of such protuberant devices/positioning mechanisms can be specialized to provide alignment for an imaging system of the present principles and can emerge more proximally.

### Radiating Extensions

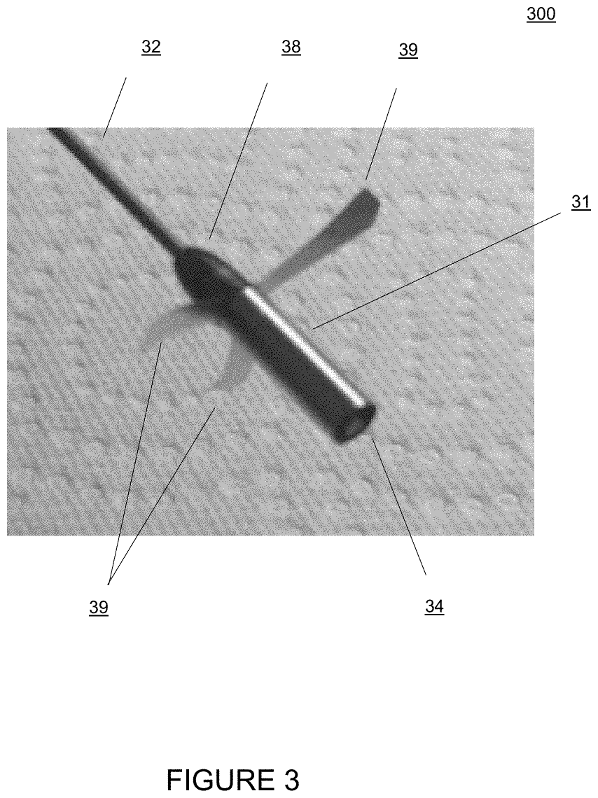

FIG. 3 depicts a high-level block diagram of an imaging system 300 including alignment extensions in accordance with an alternate embodiment of the present principles. The imaging system 300 of FIG. 3 illustratively comprises an imaging capsule 31, a tether 32, a bullet weight 38, a camera lens 34 and radiating alignment extension(s) 39 (illustratively three alignment extensions). Although in the embodiment of FIG. 3 the imaging system 300 includes three radiating alignment extensions 39, in alternate embodiments, an imaging system of the present principles can comprise more or less radiating alignment extensions 39.

In the embodiment of FIG. 3, the radiating alignment extensions take the form of radiating arms. In some embodiments, the radiating alignment extension(s) 39 can include sponge material, sheets and numerous other possibilities. For example, flexible arms provide key advantages in terms of fabrication, control of properties and ease in configuration. The embodiment of FIG. 3 depicts 3 radiating arms, although the number can be more or less.

In the embodiment of FIG. 3 the bullet weight 38 can include a bore (not shown) allowing passage of the tether 32 into the imaging capsule 31. In addition, in the embodiment of FIG. 3, the radiating alignment extension(s) 39 are positioned between a flat distal end of the bullet weight 38 and the flat proximal end of the imaging capsule 31. In some embodiments, the entire structure can be formed from a flat polymer sheet that can be formed through common practices such as stamping, laser cutting or 3-d printing.

In some embodiments, the radiating alignment extension(s) 39 are very flexible and exert a weak stabilizing force. The radiating alignment extension(s) 39, illustratively each comprising three fins can be both flexible and elastic with a good retention of shape. As such, if the imaging system 300 passes through a cylindrical tube (esophagus), the radiating alignment extension(s) 39 can collapse sufficiently to enable the imaging system 300 to progress in a descent through the esophagus.

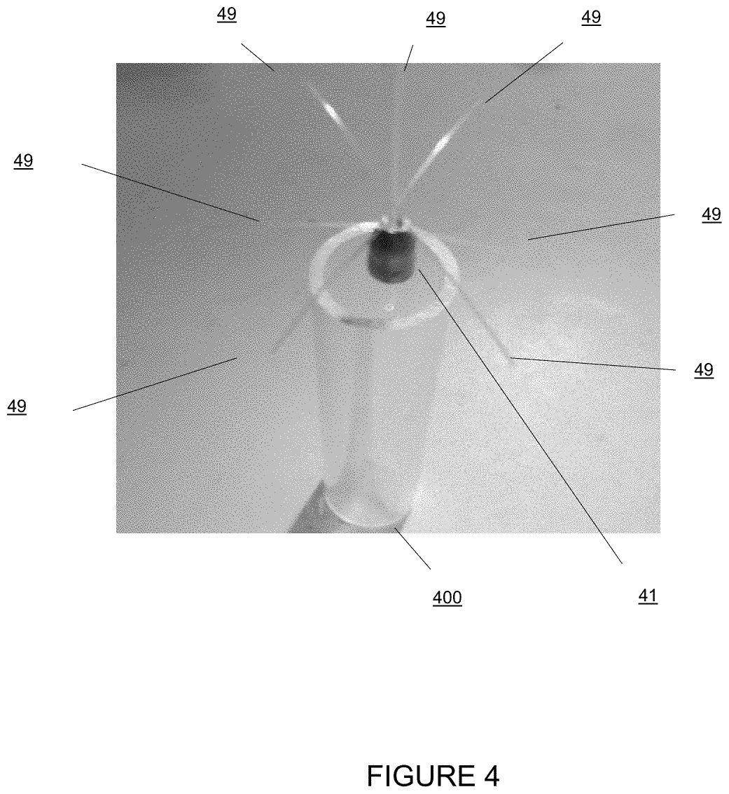

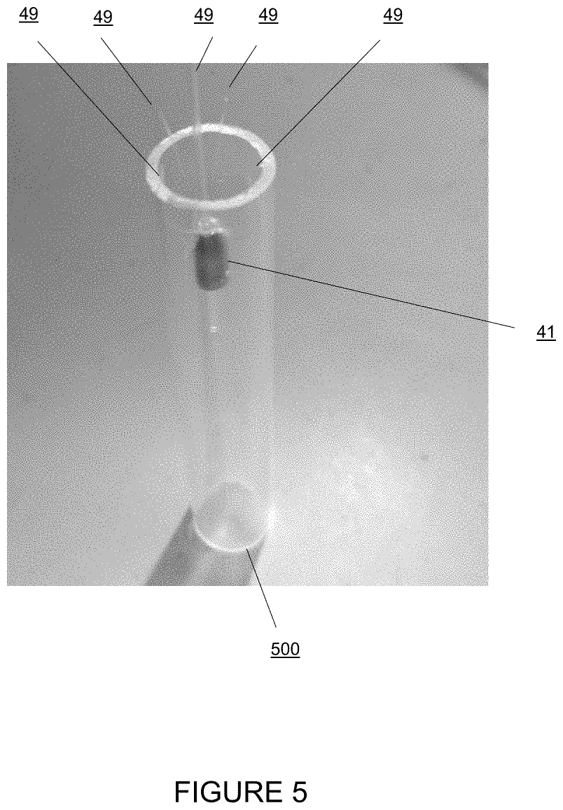

FIG. 4 and FIG. 5 depict respective embodiments of an imaging system comprising radiating alignment extension(s), such as the imaging system 300 of FIG. 3, at different stages of descension down a tube 400, 500. In the embodiment of FIG. 4 a plurality of 0.007-inch inner diameter and 0.014-inch outer diameter polyurethane tubules 49, each 1.5″ long, are attached at their centers to a proximal end of the tapered end portion of the imaging capsule 41, measuring 3/16 of an inch and hanging over a polyurethane tube 400. In the embodiment of FIG. 4, the polyurethane tubules 49 extend ¾″ from a central axis of the imaging capsule 41 and suspend the imaging capsule 41 over the tube 400.

In the embodiment of FIG. 5, the imaging capsule 41 is depicted having descended down the tube 500 with an inner diameter of ½ inch. In the embodiment of FIG. 5, the polyurethane tubules 49 substantially dampen any pendulum motion and suspend the imaging capsule 41 in place in the tube 500. In the embodiment of FIG. 5, the polyurethane tubules 49 illustratively radiate at approximately 60-degree angles. The embodiment of FIG. 5 depicts how the polyurethane tubules 49, which serve to dampen pendulum motion, are nonetheless flexible enough to allow the weight to easily drop down the ½″ ID tube 500. The polyurethane tubules 49 are comprised of material that bends substantially when supporting the full weight (e.g., a few grams) of the imaging capsule 41 and such flexibility is important for allowing passage through other passages, such as the esophagus and is also important for avoiding discomfort within a patient's mouth.

Although in the embodiments of FIG. 4 and FIG. 5 the radiating alignment extension(s) 49 are depicted as comprising polyurethane tubes, in alternate embodiments, the radiating alignment extension(s) 49 need not be tubes, and they need not be constant cross-sectional shape. In addition, in some embodiments the radiating alignment extension(s) can be curved in a hook shape that extends backwards (proximally). The radiating alignment extension(s) of the present principles provide greater comfort and a better centering of an imaging system within the lumen as restoring forces can become greater as the arms bend more.

In embodiments of the present principles, a compromise must be struck between the forces needed for alignment and requirements that include easy passage down the lumen and comfort. For the radiating alignment extension(s) of the present principles, lengths can vary and some extensions can be long and others short within the same device. In addition, the widths of the radiating alignment extension(s) can vary—perhaps being quite wide to the point of connecting with adjacent arms. Even further, the radiating alignment extension(s) might be connected, as in a snow-flake pattern—with struts.

### Radiating Extensions Details

In accordance with the present principles, the radiating alignment extension(s) can comprise many materials, such as laminates, which can be used to tune the properties of the radiating alignment extension(s). In some embodiments, tuning can be accomplished by adjusting a cross-sectional shape of the material. In some embodiments, gradient or laminate materials can also be implemented. Some embodiments use polyurethane because polyurethane has a balance between stiffness, flexibility, and shape memory, and has well established biocompatibility properties. That is, polyurethane while not unique is a highly suitable material for alignment extensions. Polyurethane can be fabricated into thin sheets, tubes and filaments and is weakly elastic with good memory retention.

From a functional viewpoint, the forces required to stabilize an imaging capsule of an imaging system of the present principles can be quite small. In most humans, an esophagus measures approximately 2-2.5 cm in diameter, and therefore radiating alignment extension(s) should be in the range of 1 to 1.5 cm in length. From a comfort perspective, it is desirable to have the radiating alignment extension(s) yield as little sensory stimulation as possible. Furthermore, if the esophageal lumen is partially constricted, it is desirable that the weight of the imaging system overcome any support or friction forces exerted by the radiating alignment extension(s). Therefore it is desirable to have the radiating alignment extension(s) apply as little force as necessary for alignment and no more, and that the radiating alignment extension(s) be easily bendable. Polyurethane possesses properties consistent with such requirements.

In some embodiments, polyurethane tubing can comprise less than a 1 mm outer diameter, and alternatively long thin filaments cut from flat polyurethane sheet 1/32-inch thick. The filaments cut from the sheets have the opportunity for cost reduction and refinement of the cross-sectional area. For example, the cut of filaments of the radiating alignment extension(s) can be tapered or otherwise altered along their length to yield controlled bending along their length.

Consider an imaging capsule of an imaging system of the present principles suspended in an esophagus having a diameter of about 2.5 cm. The imaging capsule can have a diameter of about 0.5 cm diameter. Therefore, in some embodiments radiating alignment extension(s) of the present principles are required to extend at least 1.0 cm radially. In some embodiments, three or more radial extensions will be sufficient to centrally align the imaging capsule in the esophagus.

In some embodiments, the embodiment of the imaging system 300 of FIG. 3 can be modified to use two long pieces of polyurethane filament cut from 1/32″ sheet, about 1.25″ total length each. The radiating alignment extension(s) can be formed into a regular “X” shape and positioned between the tube and the bullet weight, to yield radial extensions, for example every 90 degrees. In some embodiments, UV cured adhesive can be added for reinforcement and sealing, however, adhesive contamination can result in poor flex qualities for the radiating alignment extension(s). Therefore, in some embodiments each filament can be placed into two or more holes within the bullet weight or other tapering structure. The holes can end blindly in the center or allow the passage of the radiating alignment extension(s) such that one long alignment extension(s) can provide support for both sides of the imaging capsule of an imaging system of the present principles. The locking of the polyurethane radiating alignment extension(s) inside the holes need not be very tight—and a myriad of mechanisms such as locking bulges can be devised to secure the radiating alignment extension(s), as the threads or filaments can be stretched as they are passed through the holes. The absence of adhesive yields excellent mechanical properties, enabling as an option for a palatant to slide over and collapse the radiating alignment extension(s), allowing the radiating alignment extension(s) to spring back into the radial pattern when the palatant is released.

In some embodiments, the palatant can be made to not slide over the radiating alignment extension(s). If the radiating alignment extension(s) extend from the most proximal portions of the imaging capsule, for example near a taper point, then the palatant can extend for nearly the full length of the imaging capsule while still allowing the radiating alignment extension(s) to extend.

The radiating alignment extension(s) can be fabricated and attached using a plurality of methods and materials including polyurethane, silicone and even metal. Fabricated elements can be formed from tubing, extrusions and stamped or laser cut from film. Attachment methods for the radiating alignment extension(s) can include bur are not limited to adhesives, thermal bonding or press-fitting.

### Alignment Loops

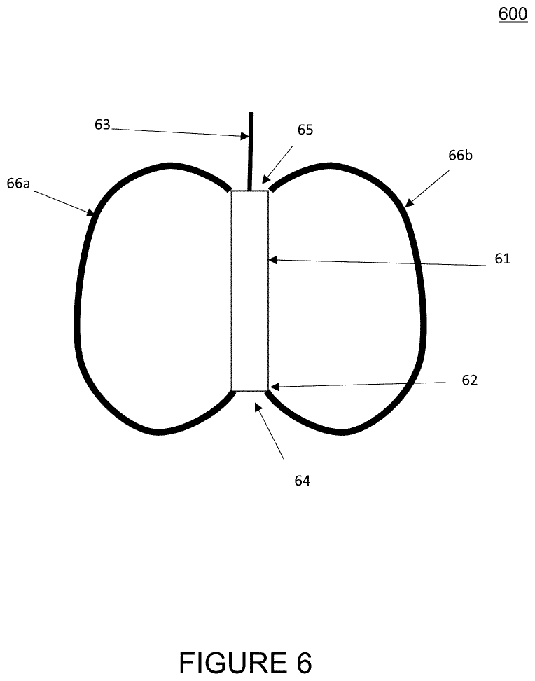

FIG. 6 depicts a high-level block diagram of an imaging system 600 including alignment loops in accordance with an alternate embodiment of the present principles. The imaging system 600 of FIG. 6 illustratively comprises an imaging capsule 61, a tether 63, a camera lens 64 and two alignment loops 66a and 66b that extend from the imaging capsule 61. In the embodiment of FIG. 6, the two alignment loops 66a and 66b originate downward and outward from a distal end 62 and loop back to a proximal end 65 of the imaging capsule 61. Each of the two alignment loops 66a and 66b can be attached to the imaging capsule 61 in two or more locations. In some embodiments, alignment loops of the present principles, such as the two alignment loops 66a and 66b of FIG. 6, can be comprised of polyurethane. That is, some embodiments use polyurethane because polyurethane has a balance between stiffness, flexibility, and shape memory, and has well established biocompatibility properties.

In the embodiment of FIG. 6, it can be desirable to increase or decrease the stiffness of at least one of the two alignment loops 66a and 66b where they emerge from the distal end 62 of the imaging capsule 61. This can be accomplished by varying the cross-sectional shape or thickness of the two alignment loops 66a and 66b. When a palatant (not shown) that can cover the two alignment loops 66a and 66b is released, the two alignment loops 66a and 66b become free to recover their shape and can serve as alignment devices. As with other alignment extension approaches, in some embodiments three or more alignment loops can be required to provide a degree of alignment stability.

In some embodiments, the two alignment loops 66a and 66b can also function as drain wicks of the present principles. In such embodiments, the angle each loop makes from the distal end 62 of the imaging capsule 61 can be controlled so as to provide the needed downward pathway for liquid while remaining clear from the field of view of the camera lens 64 of the imaging capsule 61. Because polyurethane and many other candidate materials are not hydrophilic, a drain wick portion of the two alignment loops 66a and 66b would require a hydrophilic coating or added hydrophilic substrate layer.

In some embodiments, alignment loops of the present principles, such as the two alignment loops 66a and 66b of FIG. 6, can comprise a spiral configuration around the imaging capsule 61, like a spring that turns back on itself, for example from the proximal to distal end of the imaging capsule 61. The compressed form of the spring-like alignment loops will have a smaller diameter with more wrapping around the imaging capsule. The material properties and form of the spring-like alignment loops can be tuned so as to provide an easy means to wrap the loop (or loops) around the imaging capsule when a palatant is applied over the imaging system, with a return to the recovered shape of the spring-like alignment loops when the palatant is released.

Alignment Integrated with Drain Wick

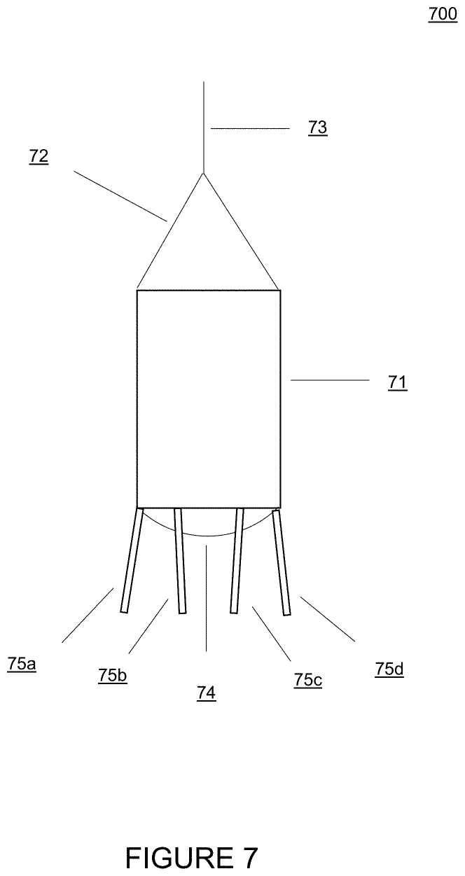

FIG. 7 depicts a high-level block diagram of an imaging system 700 including alignment extension which can also function as drain wicks in accordance with an alternate embodiment of the present principles. The imaging system 700 of FIG. 7 illustratively comprises an imaging capsule 71, a tapered end portion 72, a tether 73, a camera lens 74 and illustratively, four drain wicks/alignment extensions 75a, 75b, 75c, and 75d. Although the embodiment of the imaging system 700 of FIG. 7 is depicted as comprising four drain wicks/alignment extensions 75a, 75b, 75c, and 75d, in alternate embodiments of the present principles, an imaging system of the present principles can comprise more or less drain wicks/alignment extensions.

An essential property of a drain wick of the present principles is that the drain wick be able to provide hydrophilic pathways that lead downward from the face of an imaging capsule. This differs from alignment extensions which typically radiate outward. However, the properties of a drain wick of the present principles and an alignment extension of the present principles can be combined to form at least one drain wick/alignment extension in accordance with the present principles. For example, in FIG. 7 the drain wicks/alignment extensions 75a, 75b, 75c, and 75d comprise hydrophilic properties and are attached to the bottom of the imaging capsule 71 near the camera lens 74 to wick moisture away from the camera lens 74. The drain wicks/alignment extensions 75a, 75b, 75c, and 75d also provide rigidity and stability for enabling a positioning of the imaging system 700 and can comprise properties of the alignment extensions as described above with reference to the imaging system 600 of FIG. 6.

In some embodiments, drain wicks/alignment extensions of the present principles can comprise loops. In such embodiments, each loop can originate as an extension from the imaging capsule 71, loop downward and then bend inward and up to meet a tip of the imaging capsule 71. Loop drain wicks/alignment extensions of the present principles can provide a controllable amount or rigidity and stability. In such embodiments, the drain wicks/alignment extensions loops material can have a degree of shape memory while being flexible enough to be folded or spun around the imaging capsule 71 in instances when a palatant is slid over the imaging capsule 71. In such embodiments, the drain wicks/alignment extensions loops simply compress under the palatant resulting in parts of the drain wicks/alignment extensions loops sticking out at one or both ends of the palatant. In some embodiments, the drain wicks/alignment extensions loops of the present principles can be configured to fold in a way that winds around an imaging capsule when the palatant is applied. This configuration will leave minimal amounts of the drain wicks/alignment extensions loops extending from either end while the palatant is in place.

### Releasable Alignment System

While much of the disclosure of the present principles focuses on how an optimum tradeoff can be engineered between related goals of comfort and ease of swallowing versus alignment performance, alternate embodiments of the present principles enable an alignment extension to be collapsed for initial swallowing, and then deployed thereafter via expansion into the form suitable for maintaining alignment.

In some embodiments, a band can be wrapped around collapsed alignment extensions to keep the alignment extensions in the collapsed form. When the band is released, the alignment extensions can then spring into a more radial pattern. In some embodiments, the band can be made of water-soluble material—where the material takes sufficient time to dissolve so as to allow the passage of the device beyond the palate before the material dissolves. In alternate embodiments, another approach can be to use a material that melts at body temperature, such as gelatin, to keep the alignment extensions in the collapsed form (i.e., via a sleeve or band). After being swallowed, the material can melt and the alignment extensions can then spring into a more radial pattern.

In alternate embodiments, another approach to releasing the collapsed alignment extensions can include to integrate a material to hold down the alignment extensions that changes state during the course of deployment. In some embodiments, the material can form a strut or other member that holds the alignment extension(s) in the collapsed (undeployed) configuration. The strut can then be disengaged to release the alignment extensions into a more radial pattern.

Described embodiments of alignment extensions of the present principles enable the alignment extensions to change state from a collapsed to a deployed form. Alternate embodiments of the present principles can include the use of pullies and other mechanisms for collapsing and releasing the alignment extensions of the present principles.

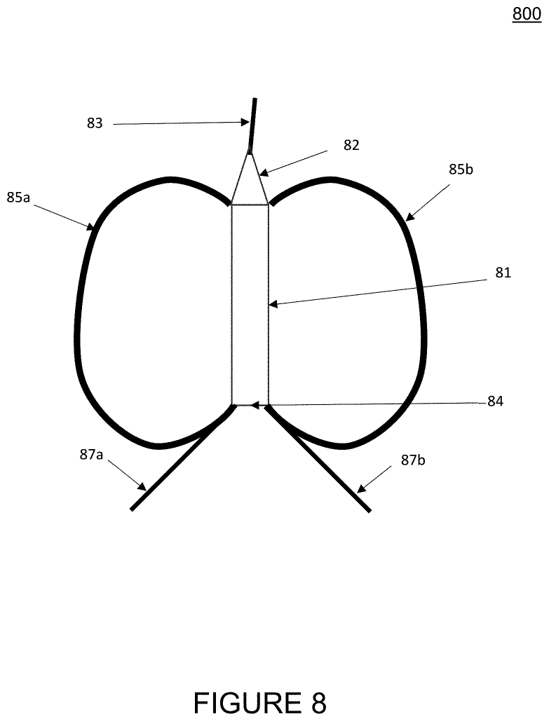

FIG. 8 depicts a high-level block diagram of an imaging system 800 including drain wicks attached to looped alignment extensions in accordance with an alternate embodiment of the present principles. The imaging system 800 of FIG. 8 illustratively comprises an imaging capsule 81, a tapered end portion 82, a tether 83, a camera lens 84, two drain wicks 87a 87b, and looped alignment extensions 85a and 85b.

The embodiment of FIG. 8 depicts an embodiment of how drain wicks of the present principles, such as the two drain wicks 87a and 87b can be directly related to alignment extensions of the present principles, such as the looped alignment extensions 85a and 85b. In the imaging system 800 of FIG. 8, the tether 83 inserts into the imaging capsule 81 via the tapered end portion 82. Extending from the junction of the tapered end portion 82 and the imaging capsule 81 are the looped alignment extensions 85a and 85b which are illustratively depicted as coplanar. In the embodiment of FIG. 8, the looped alignment extensions 85a and 85b extend around and insert into the edge of the imaging capsule 81 at a specified angle. In the embodiment of FIG. 8, attached to each looped alignment extensions 85a and 85b, and extending at specified angles are the two drain wicks 87a and 87b. In the embodiment of FIG. 8, the drain wicks 87a and 87b extend from and come in contact with a face of the imaging capsule 81, the position and orientation of the drain wicks 87a and 87b being maintained through each of the respective looped alignment extensions 85a and 85b. Although in the embodiment of FIG. 8, only two drain wicks and respective looped alignment extensions are depicted, in alternate embodiments of the present principles other numbers of drain wicks and looped alignment extensions can be included in an imaging system of the present principles.

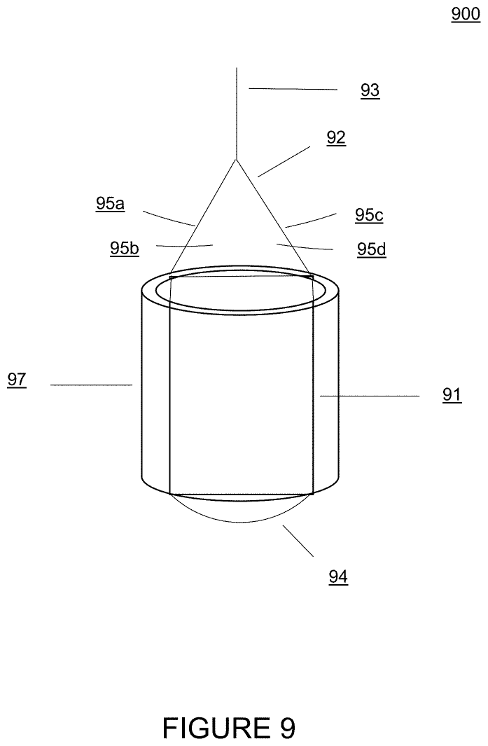

FIG. 9 depicts a high-level block diagram of an imaging system 900 including proximally located alignment extensions in accordance with an alternate embodiment of the present principles. The imaging system 900 of FIG. 9 illustratively comprises an imaging capsule 91, a tapered end portion 92, a tether 93, a camera lens 94, a palatant 97, and alignment extensions 95a, 95b, 95c and 95d extending radially from the tapered end portion 92.

In the embodiment of FIG. 9 the alignment extensions 95a, 95b, 95c and 95d can be implemented as very fine flexible arms, yielding less discomfort to a patient when swallowing the imaging system. However, an unusual mouthfeel due to the alignment extensions 95a, 95b, 95c and 95d can be unavoidable if the alignment extensions 95a, 95b, 95c and 95d freely extend radially. In some embodiments, mitigations involve tapering the arms to very fine points or bending them proximally so that that sharp points do not impinge on sensitive tissue. Such mitigation can be implemented using soft polymers and are within the scope of the present principles.

In the embodiment of the imaging system 900 of FIG. 9, the tether 93 attaches to the imaging capsule 91. The tapered end portion 92 provides a transition between the cylindrical form of the imaging capsule 91 and the tether 93. The palatant 97 attaches to the imaging capsule 91. When inside the lumen of the esophagus of a human, the alignment extensions 95a, 95b, 95c and 95d touch the walls of the esophagus, helping to maintain alignment of the imaging capsule 91 along the central axis of the lumen. When freely hanging, the imaging capsule 91 can be prone to oscillatory pendulum motion, resulting in motion of the imaging elements of the camera lens 94. This motion results in apparent motion of the image which can be highly distracting and difficult to remove via motion compensation algorithms if it is of too great an amplitude. The alignment extensions 95a, 95b, 95c and 95d serve to dampen these oscillations. In this embodiment, the alignment elements protrude from the proximal end (i.e., tapered end portion 92) of the imaging capsule 91 and are therefore clear of the attached palatant 97.

In some embodiments, a palatant or similarly tubular formed object might be slid over the arms and tether, forcing the arms against the tether. This object can act as the palatant 97, a second palatant (not shown) or a different object dedicated for this purpose and can be referred to as a “capture sleeve”. As partially described above, the capture sleave can be detachable through a number of means: melting, dissolving or mechanical action on an attached tether.

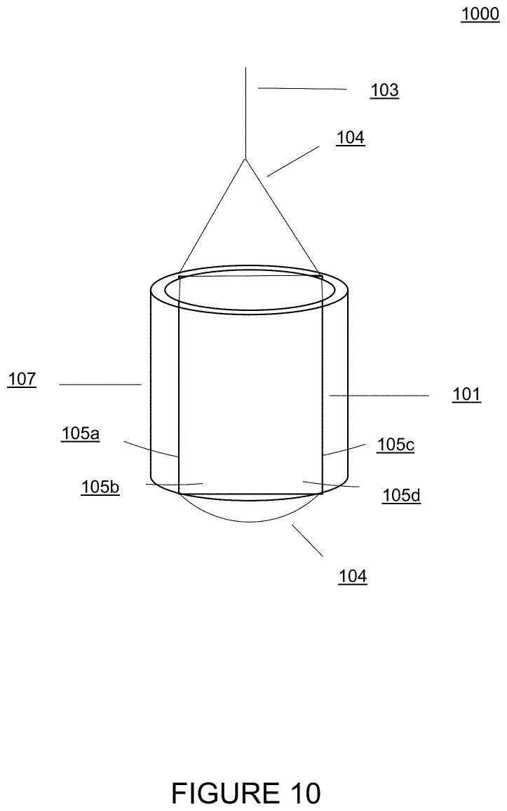

FIG. 10 depicts a high-level block diagram of an imaging system 1000 including distally located alignment extensions in accordance with an alternate embodiment of the present principles. The imaging system 1000 of FIG. 10 illustratively comprises an imaging capsule 101, a tapered end portion 102, a tether 103, a camera lens 104, a palatant 107, and alignment extensions 105a, 105b, 105c and 105d extending radially from the end of the imaging capsule 101 near the camera lens 104.

In the embodiment of FIG. 10, the alignment extensions 105a, 105b, 105c and 105d are distal on the imaging capsule 101 enabling the extended alignment extensions 105a, 105b, 105c and 105d to coexist with the attached palatant 107 without interference. In the embodiment of FIG. 10, the tether 103 attaches to the imaging capsule 101 in the presence of the tapered end portion 102. In FIG. 9, the palatant 107 is attached to the imaging capsule 101. At the distal end of the imaging capsule 101 are attached the alignment extensions 105a, 105b, 105c and 105d. In the embodiment of FIG. 10, the alignment extensions 105a, 105b, 105c and 105d are implemented to maintain the position of the palatant 107, preventing the palatant 107 from sliding distally forward. In some embodiments, the palatant 107 of FIG. 10 can be slid past the imaging capsule 101 enough to enable the alignment extensions 105a, 105b, 105c and 105d to extend to their active positions. The palatant 107 can then be slid forward (distally) into a final position. Although the embodiment of FIG. 10, drain wicks are not depicted, drain wicks can be added to the palatant of FIG. 10 as previously described in other embodiments.

The drain wick and alignment extensions of the present principles can be attached directly to, for example, the imaging capsule of an imaging system of the present principles through various bonding techniques. In alternate embodiments, the drain wick and alignment extensions of the present principles can be integrated into a structure that can be separately applied. For example, in some embodiments, the drain wick and alignment extensions of the present principles can comprise arm structures that are attached to a tubular sleeve that, in turn, can then slip over an imaging capsule of an imaging system and can remain attached by contraction on wetting, tapered fit or other means. Such a tubular sleeve can be molded and include a wicking lip structure or bezel that closely affixes to the edge of a camera lens of an imaging system of the present principles. A ring of wicking material on the tubular sleeve can extend 360 degrees and encroach the camera lens only enough to allow the minimum necessary optical aperture clearance.

In some embodiments, an imaging system of the present principles can include a palatant, edible or otherwise, formed around the tubular sleeve, the entire assembly consisting of one unit attachable to an imaging capsule of an imaging system of the present principles. In some embodiment, by sliding the palatant downward (distally) into a final position, portions of the alignment extensions of the present principles can extend down from the camera lens—in some embodiments, enabling alignment extensions having wicking properties to wick properly.

While gravity has been emphasized for removing excess fluid from a drain wick of the present principles, thereby enabling the drain wick to continue to function, other approaches are within the concepts of the present principles. For example, in some embodiments, the tubular sleeve can be thickened and coated with a water impermeable layer, so that the only way fluid can reach the tubular sleeve is via the drain wick or alignments extensions having wicking properties. That is, in some embodiments, the tubular sleeve can function as a reservoir that can sequester fluid.

In the drawings, specific arrangements or orderings of schematic elements can be shown for ease of description. However, the specific ordering or arrangement of such elements is not meant to imply that a particular order or sequence of processing, or separation of processes, is required in all embodiments. In general, schematic elements used to represent instruction blocks or modules can be implemented using any suitable form of machine-readable instruction, and each such instruction can be implemented using any suitable programming language, library, application-programming interface (API), and/or other software development tools or frameworks. Similarly, schematic elements used to represent data or information can be implemented using any suitable electronic arrangement or data structure. Further, some connections, relationships or associations between elements can be simplified or not shown in the drawings so as not to obscure the disclosure.

This disclosure is to be considered as exemplary and not restrictive in character, and all changes and modifications that come within the guidelines of the disclosure are desired to be protected.

1. An imaging system, comprising:
a tether;
a tapered end portion coupling the tether to an imaging capsule;
the imaging capsule comprising a camera lens; and
at least one fluid wicking element, positioned on the imaging system proximate to the camera lens of the imaging capsule to enable the fluid wicking element to wick liquid away from the camera lens.

2. The imaging system of claim 1, wherein the fluid wicking element uses at least one of a wicking property of a material of the fluid wicking element or gravity to wick liquid away from the camera lens.

3. The imaging system of claim 1, wherein the wicked fluid can accumulate on the fluid wicking element and be drawn off of the fluid wicking element by gravity.

4. The imaging system of claim 1, wherein fluid accumulated on the fluid wicking element is drawn off by contact with a surrounding absorbent material.

5. The imaging system of claim 1, wherein the fluid wicking element comprises a drain wick.

6. The imaging system of claim 1, wherein the fluid wicking element further functions as an alignment element for the imaging system.

7. The imaging system of claim 1, further comprising at least one alignment element for aligning the imaging system when inserted in an opening.

8. The imaging system of claim 7, wherein the opening comprises a human esophagus.

9. The imaging system of claim 7, wherein the at least one alignment element comprises three alignment elements to center the imaging system in the opening.

10. The imaging system of claim 7, wherein the at least one alignment element is configured as a loop having at least two attachments to components of the imaging system.

11. The imaging system of claim 7, wherein the at least one alignment element comprises fluid wicking properties.

12. The imaging system of claim 1, wherein the fluid wicking element comprises at least one of a hydrophilic or lubricious coating.

13. An imaging system, comprising:
a tether;
a tapered end portion coupling the tether to an imaging capsule;
the imaging capsule comprising a camera lens; and
at least one alignment element attached on the imaging system to enable a positioning of the imaging system in an opening.

14. The imaging system of claim 13, further comprising a palatant, wherein the at least one alignment element is attached to the palatant.

15. The imaging system of claim 13, wherein the at least one alignment element comprises fluid wicking properties.

16. The imaging system of claim 15, wherein the at least one alignment element is positioned on the imaging system proximate to the camera lens of the imaging capsule to enable the at least one alignment element comprising the fluid wicking properties to wick liquid away from the camera lens.

17. The imaging system of claim 13, further comprising a restraining element to collapse the at least one alignment element.

18. The imaging system of claim 17, wherein the at least one alignment element is collapsed by the restraining element to enable placing the imaging system into an opening.

19. The imaging system of claim 18, wherein the restraining element is removed and the at least one alignment element expands after entering the opening.

20. The imaging system of claim 18, wherein the at least one alignment element is constructed of materials having such fine proportions that forces exerted on surfaces of the opening by the at least one alignment element are insufficient to impede the passage of the imaging system through the opening when the imaging system is pulled by gravity.
